(19) United States
(12) Patent Application Publication
(10) Pub. No.: US 2009/0263467 A1
(43) Pub. Date: Oct. 22, 2009

(54) COMBINATION DRUG THERAPY USING ORALLY DISSOLVING FILM OR ORALLY DISINTEGRATING TABLET DOSAGE FORMS TO TREAT DRY MOUTH AILMENTS

(76) Inventor: Hemant Narahar Joshi, Parsippany, NJ (US)

Correspondence Address: HEMANT N. JOSHI/TARA ENTERPRISES, 41 LEAH WAY, PARSIPPANY, NJ, 07054, US

(21) Appl. No.: 12/107,039

(22) Filed: Apr. 21, 2008

(51) Int. Cl. A61K 9/70; A61K 31/439; A61K 38/43; A61K 31/4178; A61K 36/31

(52) U.S. Cl. 424/443; 514/305; 424/94.1; 514/397; 424/755

(57) ABSTRACT

A combination drug therapy can be administered to patients to treat xerostomia or dry mouth or Sjogren's syndrome in the form of orally dissolving film or orally disintegrating tablets. Some of the drugs could be synthetic origin and some drugs are obtained from natural sources.

### BACKGROUND OF THE INVENTION

Dryness of mouth/Xerostomia may result from a decreased production of saliva. Saliva keeps mouth healthy by providing lubrication, pH maintenance, and provides minerals such as calcium, fluoride and phosphorous. Saliva cleans the mouth cavity. It helps in digesting and swallowing food. Saliva also prevents infections by controlling bacteria and fungi levels in mouth. Approximate daily output of saliva is approximately one liter/day. Dry mouth may cause a burning effect, fungal/bacterial infections and overall it may decrease the quality of life.

There could be various causes of the dry mouth symptom. One of the key causes could be the side effects of various modern medicines such as drugs to treat pain, allergy/cold, obesity, epilepsy, hypertension, and sedatives. Hyposalivation can occur as a side effect of certain disease conditions such as infections, HIV, diabetes, hypertension etc. Certain medical treatments such as radiation therapy also can cause dryness of mouth. Excessive sweating, vomiting, blood loss, diarrhea, fever etc. can also produce dryness of mouth. Surgical removal of salivary glands can produce less saliva resulting in dry mouth. People who smoke, and chew tobacco may experience dry mouth. Although dry mouth is not a life-threatening disease, it could psychologically irritate patients and can cause other secondary symptoms. Dry mouth can result into frequent thirst, sores in mouth, cracked lips, dry/red tongue, bad breath and burning/tingling sensation in the mouth cavity.

Many drugs have been invented in the recent years to treat dry mouth and serving our community well. Several types of drug delivery systems such as lozenges, mouthwashes, buccal patches etc. have been employed to deliver these drugs. Biotene is used to treat dry mouth which contains antimicrobial enzymes found naturally in human saliva—glucose oxidase, lactoperoxidase, lactoferrin and lysozyme. It is used in the form of toothpaste, mouth gums, mouthwash and moisturizing gel. Pilocarpine (Salagen, 5 or 7.5 mg, MGI Pharma) and Cevimeline Hydrochloride tablets (Evoxac, 30 mg, Daiichi Pharmaceutical Corporation) are meant for oral administration to treat dry mouth. Edible organic acids such as citric acid, malic acid etc. stimulate salivation. Similar effect is observed with polyalcohol such as glycerol. Recently, calcium and phosphate ions were also shown to induce salivation and were incorporated in a mouthwash formulation. Meswak extract has a historical value and it is commonly used in toothpastes.

Orally dissolving films (ODF), as a delivery system, is becoming popular. US patent application # 20080008743 has been filed to deliver memantine, a drug for childhood behavioral disorders and Alzheimer's disease using ODF technology. US Patent Application # 20070154542 used ODFs to deliver non-steroidal anti-inflammatory drugs and acid inhibitors. US Patent Application # 20060205629 used medium to high bloom gelatin as the major component of edible dissolving gelatin strips. US Patent Application # 20060198873 produced ODF with enteric polymer and alkaline buffer to deliver nicotine. Multilayer ODF with at least one drug in a film have been listed in the U.S. Pat. No. 7,332,230. Multilayer films are also prepared in the transdermal delivery systems and thus, the technology is well established. U.S. Pat. No. 7,182,964 describes the preparation of ODF containing a xanthone. The edible films of pullulan were made with antimicrobially effective amounts of the essential oils, which were effective at killing the plaque-producing germs that cause dental plaque, gingivitis and bad breath (U.S. Pat. No. 7,025,983). U.S. Pat. No. 6,709,671 described water-soluble films for oral administration containing pharmaceutically active ingredients. US Patent Application # 20080050422 claimed orally dissolving films with at least one drug and would dissolve in 3.5 seconds upon administration of a fluid while film was in the mouth. The absorption of drug was observed to be higher when the film was administered with a fluid compared to when the film was administered without the fluid. In another U.S. patent application, the drug particles in the orally dissolving films were coated for taste masking or controlled-release (US Patent Application # 20080044454). Paladin Labs, Inc. filed a patent for Thinsol™, a water-soluble enzymatically digested film of carboxymethoxy cellulose, which is four times thicker than normal ODF's and in this product, the drug loading can be up to 60% of the film weight. The film can be prepared at low temperature so that the heat-sensitive drugs can be loaded. MonoSolRx is working on ODF products containing active pharmaceutical ingredients such as Zolpidem (sleep disorder), Ondansetron HCl (Nausea/vomiting), Donepezil HCl (Alzheimer's disease) arid Escitalopram Oxalate (Anti-depressant). Novartis has announced line extensions of their products, Triaminic and Theraflu, as ODF. Zengen has developed an oral film containing local anesthetic benzocaine for the treatment of sore throat. Another product, Chloraseptic Relief Strips contains benzocaine, a local anaesthetic, as an active ingredient and menthol. Bioprogress, a UK based company, acquired the ODF business of AquaFilm, a Florida based company in 2004. Applied Pharma Research and Labtec showed a bioequivalence for their orally dissolvable film of ondansetron and GlaxoSmithKline's ODT. Students of Johns Hopkins developed ODF's containing rotavirus vaccine, which is easy to store and transport and would not require refrigeration.

Orally disintegrating tablets (ODT) became popular much earlier than ODF. The key aspects are to manufacture tablets with satisfactory hardness, low friability and rapid disintegration in the mouth cavity. U.S. Pat. No. 7,282,217 described an ODT prepared with a water-soluble pharmaceutically active carbohydrate and water-insoluble filler and by a wet granulation method. U.S. Pat. No. 6,368,625 described an ODT formulation, which produced viscous organoleptically pleasant material which preventing the spread of insoluble materials including the drug. In the US Patent Application 20070292508, the inventors could mask the taste of an active ingredient by lipids and the silicified excipient provided desired properties for the ODT formulation. There are many formulations developed using ODT technology, but none of the formulations was for the treatment of dry mouth. Apart from the problems of low hardness and high friability, the ODTs have normally low total weights of the dosage form limiting the drug loading.

Saliva pH is 7.1 to 7.5 in normal population, but can be as low as 4.5 in cancer patients. Addition of calcium phosphate to the ODF or ODT formulations will increase the microenvironmental pH in the dosage form and may increase the pH in the mouth cavity. Calcium and phosphate ions have been reported to induce salivation. Cytogen markets Caphosol mouthwash, a product for dry mouth treating oral mucositis to be used often with chemotherapy. It contains high concentrations of calcium and phosphate ions (U.S. Pat. No. 5,993,785).

Biotene is used in the form of toothpaste, mouthwash, moisturizing gel and mouth gums and is recommended to everyone susceptible to cavities, bad breath, mouth sores, and gum diseases. It is also used to treat dry mouth and contains antimicrobial enzymes found naturally in human saliva—glucose oxidase, lactoperoxidase, lysozyme and lactoferrin.

Pilocarpine is a choline ester miotic (cholinergic parasympathomimetic agent) and can increase secretion of exocrine glands such as sweat, saliva and lacrimal. Due to short half-life (0.76 hours), frequent dosing of pilocarpine is needed. The dose of pilocarpine should not exceed 30 mg per day. Pilocarpine chewing gums are available, which provide the active ingredient as well as the mechanical action of chewing which help salivation. US Patent Application # 20060029665 is for the chewing gum of pilocarpine (dose 2 to 5 mg), wherein the pH is altered to increase absorption of the active ingredient. U.S. Pat. No. 4,209,505 was approved for the use of pilocarpine (0.025% to 1%) in mouthwash to treat dry mouth. It is bitter in taste and therefore, the taste is masked by sweeteners.

Cevimeline is a cholinergic agonist which stimulates muscarinic receptors and is used to treat Sjogren syndrome (an autoimmune disorder in which immune cells attack and destroy exocrine gland that produce tears and saliva). Hachiazule and xylocaine gargles therapy was cited for cancer patients to treat stomatitis (inflammation of mouth). Cevimeline was added to the treatment and was observed to be effective against the dry mouth issues. In the US Patent Application # 20060062787 for the treatment of Sjogren's syndrome, one of the active ingredients was cevimeline.

Citric acid, malic acid and other edible organic acid (tartaric, fumaric, phosphoric, oxalic acids) are commonly used in the mouth moistener formulations. U.S. Pat. No. 5,658,554 used about 2% of citric acid or malic acid in aqueous vehicle for alleviating dry mouth condition. U.S. Pat. No. 5,614,207 described the addition of various edible acids including ascorbic acid in the lozenges to treat dry mouth.

Many formulations to treat dry mouth symptoms use humectants such as glycerol, sorbitol, polyethylene glycol, and xylitol in the weight range of 1 to 1 0%. Dr. Collins All White Whitening toothpaste contains 25% xylitol, peroxide and cetyl pyridinium chloride. Rain dry mouth spray used to treat xerostomia contains high concentration of Xylitol. Xylitol and lactoferrin are the active ingredients of NutraSip solution used to treat the dry mouth. NutraSip contains NutraFerrin, which is a mixture of bovine lactoferrin (a natural protective protein) and special milk proteins. Xylitol in the formulation improves the mouth feel and taste. Lactoferrin is an iron-binding glycoprotein with antimicrobial properties.

Meswak Extract has a historical value to treat mouth ailments. It is believed that Prophet Muhammad recommended its use as a Chewing stick (Kayu Sugi) and is used in Muslim population. It has been shown to have a significant antimicrobial activity especially against microbes causing bad breath. Meswak extract is commonly used in the toothpastes to clean teeth and as a mouth-freshener. Departments of Chemistry, Riyadh University, Saudi Arabia and Indiana University, Indiana, USA confirmed the anti-inflammatory and antibacterial activities of Meswak in independent studies.

Combination drug therapy is commonly used to treat many diseases. Multivitamin tablets, sulfamethoxazole-trimethoprim tablets, cough medicines including antihistamines; decongestants etc. are used in day-to-day lives. There are about 50 anti-cancer drugs and combination therapy has been proven more effective compared to single drugs. The combinations are often known by a short name such as MAID (Mesna, Adriamycin, Ifosfamide, and Dacarbazine), and AIM (Adriamycin, Ifosfamide, and Mesna). Epzicom, the HIV treatment, is a combination of Abacavir and lamivudine. Combination drug therapy is expected to continue to be the part of future drug and dosage form developments. In the U.S. Pat. No. 6,835,728, the combination of mirtazapine and gepirone was claimed to be better to treat depression. In the U.S. Pat. No. 6,960,577, olanzapine and fluoxetine were used as a combination therapy. U.S. Pat. No. 6,942,876 claimed that a combination therapy of antiepileptic compounds that demonstrated pain-alleviating property and compounds from a group of analgesics (NMDA receptor agonists, NSAIDs) decreased the frequency and severity of pain and reduced the side effects. Combination drug therapy also allows avoidance of taking multiple tablets/capsules per day, saving on the co-payments for different medicines and assurance of patient-compliance to drug therapies. It is important to prove that different drugs combined in the same dosage form are stable during manufacture and storage. In the combination dosage form, each drug should show the desired release rate from the dosage form to produce the desired rate and extent of absorption upon oral administration.

The patent application herein proposes to use combination of various drugs in the ODF or ODT dosage forms to treat dry mouth conditions. ODF's are generally made by casting films on a flat surface. Hot air is mainly used to dry the coated solutions. Joshi et al. proposed the use of microwave drying in film coating (Int. J. Pharm. 51:19-25, 1989). Malinger et al described the usage of microwave drying in coating articles (US Patent Application # 20070154639). Vacuum drying may also be used to evaporate solvents from the film solution. Sometimes it is desirable to dry the film at lower temperature due to instability of the drug or evaporation of volatile ingredients in the formulation. In such cases, application of vacuum to the system would be helpful for an effective drying.

### SUMMARY OF THE INVENTION

The present invention is about the usage of an appropriate combination of drugs known to be effective against the hypo salivation in mouth and administered in the form of an ODF or ODT dosage forms. This combination dosage form will be used for the treatment of dry mouth/xerostomia or Sjogern's syndrome. Some of the drugs proposed could be of synthetic origin and some could be naturally occurring. The actives to be used in this combination drug therapy include biotene (glucose oxidase, lactose peroxidase, and lysozyme), an edible organic acids, polyalcohols, sugars, meswak extract, pilocarpine, cevimeline, calcium ions, phosphate ions, essential oils and all the combinations thereof. Pilocarpine and cevimeline can be used as a free base or as a suitable salt. It is observed for some of the synthetic drugs that one needs to take treatment for a long time to get a significant pharmacological effect. The combination drug therapy might produce an effect in a short time. Both the dosage form (ODF and ODT) can be administered conveniently without water or juice and it will produce localized action in the mouth. The dosage forms can be made palatable by the addition of flavors and sweeteners. In the ODF dosage form, the drugs could be incorporated in a single layered or in a bi-layered film.

### DETAILED DESCRIPTION

The proposed invention has two types of dosage forms—ODF and ODT. The composition of the base formula and the process involved for both are commonly known to experts working in the field of pharmaceutical product development. An appropriate combination of drugs would produce the desired effects faster, allow reduction of doses for individual drugs, provide convenience to patients in terms of administration of the dosage form and produce a pleasant taste in the mouth cavity. One or more drugs from the intended list of drugs can be incorporated in the base formulation. The drug combinations can be chosen from the following list—biotene (glucose oxidase, lactoperoxidase, lysozyme, and lactoferrin), pilocarpine, cevimeline, citric acid or other edible organic acids, glycerin or other polyalcohols, and meswak extract. Because the doses of drugs would be different in different combinations, it is not possible to propose exact doses for every possible combination. One should also add various flavoring agents (strawberry, vanilla etc.) and sweeteners to produce a pleasant taste in the mouth.

The following table shows an example of the base formula for the ODF. It used hydroxypropyl methylcellulose and sodium alginate combination to prepare films, which dissolve in the mouth cavity in 10-60 seconds. Citric acid and glycerin induce salivation, but also contribute to a pleasant taste. Sodium lauryl sulfate is a surfactant and in a small amount in combination with some of the essential oils, it provides mouth cleaning/refreshing effect. Sucralose is a sweetener.

| Ingredient | Percent |
|---|---|
| Hydroxypropyl methyl | 27 |
| cellulose | |
| Sodium alginate | 43 |
| Citric acid | 7 |
| Glycerin | 20 |
| Sucralose | 0.3 |
| Sodium lauryl sulfate | 2.7 |
| Water | Quantity sufficient (Q.S.) |

A glass plate can be used for the casting of the films, which can be dried in air at room temperature, in the refrigerator or using a microwave. The temperature of product increases due to drying of film in the microwave and therefore, care must be taken to control the temperature of the product.

The ODF can be prepared as a single-layered or bi-layered film preparation. In the bi-layered film preparation, the pH in the two films could be kept acidic (pH 3 to 4) and neutral (pH 6.5 to 7.5), which would help to stabilize the drugs. The bi-layered film preparation can also be used effectively to prevent-drug-drug interactions and improve stability of the formulation.

For the film composition, water-soluble polymers are selected from the group consisting of carboxymethyl cellulose, carboxyvinyl polymers, high amylose starch, hydroxyethyl cellulose, hydroxypropyl cellulose, hydroxypropyl methylcellulose, methylmethacrylate copolymers, polyacrylic acid, polyvinyl alcohol, polyvinyl pyrrolidone, pullulan, sodium alginate, and combinations thereof.

The film composition may also contain one of the sugars from the selected group consisting of glucose, dextrose, fructose, lactose, maltose, xylose, sucrose, corn sugar syrup, sorbitol, hexitol, maltilol, xylitol, mannitol, and combinations thereof.

The film composition may contain one or more essential oils selected from the group consisting of eucalyptol, menthol, vacrol, thymol, methyl salicylate, verbenone, eugenol, gerianol and combinations thereof.

The film composition may contain a polyalcohol selected from the group consisting of glycerol, polyethylene glycol, propylene glycol, and combinations thereof.

The film composition may contain an edible organic acid from the group consisting of citric acid, malic acid, tartaric acid, fumaric acid, phosphoric acid, oxalic acid, ascorbic acid and combinations thereof.

It is important that the film should dissolve in the mouth in a short time, 10 to 60 seconds and should not produce a sticky feeling in the mouth. It should produce a pleasant taste in the mouth.

In case of orally disintegrating tablets, a suitable adsorbent should be added to the formulation to adsorb the liquid ingredients. Excipients such as a binder, disintegrant, filler etc. are used commonly in the tablet dosage forms and should be used in the ODT formulation.

In the case of both ODF and ODT dosage forms, it is possible that part of the active ingredients may be absorbed from the mouth cavity and may produce a rapid local effect. The salivary pH is neutral and pilocarpine permeability is reported to be higher in neutral pH. However, it has bitter taste and therefore, the ODF and ODT formulations containing pilocarpine as one of the active ingredients should contain a sweetener and flavoring agents.

Pilocarpine and cevimeline exist as a free base or as salts (for examples, as a hydrochloride salt). If the free base form is used in the formulation, these active pharmaceutical ingredients must be used in a fine powder form to achieve uniform distribution in the ODF and ODT dosage forms.

What is claimed is:

1. A combination drug therapy for the localized treatment of dry mouth/xerostomia or Sjogren's syndrome using orally dissolving film (ODF) or orally disintegrating tablet (ODT) delivery systems.

2. The drugs in the combination therapy delivered in the form of ODF or ODT as in claim 1 wherein the active moieties include biotene (glucose oxidase, lactose peroxidase, and lysozyme), an edible organic acid, polyalcohol, sugar, meswak extract, pilocarpine, cevimeline, calcium ions, phosphate ions, essential oils and all the combinations thereof.

3-8. (canceled)

9. The ODF or ODT combination dosage form in claim 1, wherein one of the combinations of actives is biotene, cevimeline, meswak, pilocarpine, citric acid, glycerin, calcium phosphate and xylitol.

10. A base composition of ODF in claim 1 wherein sodium alginate and hydroxypropyl methylcellulose are mixed in various proportions to prepare films.

11. An addition of a suitable adsorbant to the base composition of ODT in claim 1 wherein silicone dioxide, calcium silicate or other silicified, and non-silicified adsorbants adsorb the liquid components such as essential oils.

12. (canceled)

13. The ODF in claim 1 wherein the dosage form is a single-layered or bi-layered film.

14. (canceled)

15. Pilocarpine in claim 2 which is used in the form of a free base, hydrochloride salt or any other salt.

16. Cevimeline in claim 2 which is used in the form of a free base, hydrochloride salt or any other salt.

17. The edible organic acids in claim 2 including citric acid, malic acid, tartaric acid, phosphoric acid, fumaric acid, and ascorbic acid or combinations thereof.

18. The sugars in claim 2 which could be selected from a group consisting of glucose, dextrose, fructose, lactose, maltose, xylose, sucrose, corn sugar syrup, sorbitol, hexitol, maltilol, xylitol, mannitol, and combinations thereof.

19. The essential oils in claim 2 which could be selected from the group consisting of eucalyptol, menthol, vacrol, thymol, methyl salicylate, verbenone, eugenol, gerianol and combinations thereof.

20. The dosage form compositions as in claim 2 which contains a polyalcohol or humectant selected from the group consisting of glycerol, polyethylene glycol, propylene glycol, and combinations thereof.

21. The ODF composition as in claim 2, which contains a water soluble polymers selected from the group consisting of carboxymethyl cellulose, carboxyvinyl polymers, high amylose starch, hydroxyethyl cellulose, hydroxypropyl cellulose, hydroxypropyl methylcellulose, methylmethacrylate copolymers, polyacrylic acid, polyvinyl alcohol, polyvinyl pyrrolidone, pullulan, sodium alginate, and combinations thereof.

22-23. (canceled)
